(19) United States
(12) Patent Application Publication
(10) Pub. No.: US 2011/0027335 A1
(43) Pub. Date: Feb. 3, 2011

(54) COATED MEDICAL DEVICES

(75) Inventors: David Fortune, Leeds (GB); David John Mandley, Leeds (GB); Diane Morris, Leeds (GB); Ian Thompson, Leeds (GB); Jamie Swallow, Leeds (GB)

Correspondence Address: NIXON PEABODY LLP - PATENT GROUP, 1100 CLINTON SQUARE, ROCHESTER, NY, 14604, US

(73) Assignee: TISSUEMED LIMITED, Leeds, Yorkshire (GB)

(21) Appl. No.: 12/672,844

(22) PCT Filed: Aug. 11, 2008
(86) PCT No.: PCT/GB2008/050691

§ 371 Date: Feb. 9, 2010

(30) Foreign Application Priority Data
Aug. 10, 2007 (GB) 0715514.6

(51) Int. Cl. A61K 9/00; A61P 43/00; A61N 1/362

(52) U.S. Cl. 424/423; 424/422; 607/36

(57) ABSTRACT

An implantable medical device carries on at least part of its external surface a coating. The coating consists essentially of a terpolymer of vinyl pyrrolidone, acrylic acid and activated acrylic acid, and optionally a colouring agent.

### FIELD OF THE INVENTION

This invention relates to coated devices intended for implantation in the body in the course of surgical procedures, and to methods involving the use of such coated devices. The invention relates particularly to implantable devices useful in numerous different types of procedures that are modified by coating with tissue-adhesive, biocompatible material.

### BACKGROUND OF THE INVENTION

The implantation of devices in the body is commonplace in surgical procedures, and many such devices are known.

Possible adverse reactions to the implantation of devices may include, but are not limited to, inflammation, migration, tissue trauma, pain and post surgical adhesions, fistula formation, seroma formation, haematoma and recurrence of tissue defect.

A particular problem that may be encountered with implanted devices is that they can become dislodged from the site of application, leading to a failure of the device to perform its intended function and/or other complications such as those mentioned above. Serious complications may necessitate further surgical intervention.

This problem may be addressed by attempting to fix the device more securely in position, e.g. by the use of sutures, staples or other forms of mechanical fastener. However, this is often difficult to achieve, and moreover, such approaches introduce further drawbacks.

There is a need for improved implantable devices that are self-adhesive (or more self-adhesive, as the case may be), and thereby overcome or substantially mitigate the above mentioned problems.

Furthermore, it would be advantageous to provide a method for modifying current implantable devices to render them self-adhesive (or more self-adhesive, as the case may be), and thereby overcome or substantially mitigate the above mentioned problems.

One group of implantable devices are graft products intended principally for implantation to join or seal tissues, to reinforce weakened soft tissue and/or to assist the repair of internal wounds. There are numerous graft products on the market, particularly soft tissue and hernia grafts, which are intended to be sutured or stapled prior to application in order to keep them in place. There is clearly a need for improved graft products that are sufficiently self-adhesive that they do not require additional mechanical attachment.

Medical devices that are coated with a tissue-adhesive formulation are disclosed in the prior art. Tissue-adhesive formulations that have hitherto been proposed include viscous solutions or gels that are either manufactured in that form or are prepared immediately prior to use by mixing of the ingredients, and then applied to the medical device or tissue surface prior to implantation of the device.

Tissue-adhesive formulations of this type suffer from a number of disadvantages. If the formulation is of low viscosity, it may spread from the device or the area of application and hence be messy and difficult to apply precisely. If the formulation is more viscous, on the other hand, it may be difficult to dispense. In either case, the formulation, being prepared in hydrated form, may have a limited lifetime. It may therefore be necessary for the whole of the formulation to be used at once or discarded. Also, the preparation of formulations immediately prior to use by mixing of ingredients is obviously laborious and time-consuming. In addition to these drawbacks, the degree of adhesion between the tissue surface and the device, that is provided by such formulations, may be less than would be desired.

Coating a device with a tissue-adhesive formulation may adversely affect the physical characteristics of the device. For example, the flexibility of the coated device may be insufficient for it to conform readily to the surface to which it is applied, which may also have an adverse effect on its adhesion.

As a result of inadequate adhesion, it may be necessary to provide further reinforcement, e.g. through mechanical attachment using sutures, staples or the like. Alternatively, energy (e.g. light or heat energy) may be applied in order to initiate chemical bonding of the adhesive formulation to the underlying tissue, and hence bonding of the tissue surfaces to each other. Clearly, such approaches introduce further drawbacks. The use of mechanical fastenings such as sutures or staples is often the very thing that the use of such products is intended to replace or avoid. In many instances, the use of such fastenings is either not wholly effective (e.g. on the lung) or undesirable, as their introduction gives rise to further areas of tissue weakness. The use of external energy requires the provision and operation of a source of such energy. Such energy sources may be expensive and difficult to operate, particularly in the confines of an operating theatre or similar environment. Also, the use of external energy for attachment can be both time-consuming and (in some cases) requires significant careful judgement on the part of the surgeon, to evaluate when sufficient energy has been delivered to effect attachment without damaging the underlying tissue.

Thus, there is a need for improved implantable devices that are self-adhesive by virtue of a biocompatible, tissue-adhesive coating. In particular, it would be advantageous for said implantable device to be pre-formed and supplied ready-for-use.

The applicant's pending international application PCT/GB2005/00298 (WO2006/013337) discloses a terpolymer of vinyl pyrrolidone, acrylic acid and acrylic acid N-hydroxysuccinimide ester. The terpolymer may be used to prepare a tissue-adhesive sheet comprising a homogeneous film.

Co-pending international application PCT/GB2007/050049 discloses a device suitable for implantation in the human or animal body, which device carries on at least part of the external surface thereof a coating comprising one or more polymers with film-forming properties (e.g. poly(DL-lactide-co-glycolide)), at least part of said coating being conjoined to a layer of material comprising tissue-reactive functional groups (e.g. poly(VP-AAc-AAc(NHS)) terpolymer).

Surprising, the applicant has found that a coating of the terpolymer disclosed in the above applications can provide a significant performance benefit to an implantable device.

Thus, there have now been devised improvements to implantable medical devices that overcome or substantially mitigate the above-mentioned and/or other disadvantages of the prior art.

### BRIEF SUMMARY OF THE INVENTION

According to a first aspect of the invention, there is provided an implantable medical device, at least part of the external surface of which bears a coating consisting essentially of a terpolymer of vinyl pyrrolidone, acrylic acid and activated acrylic acid, and optionally a colouring agent.

The device according to the invention is advantageous because the tissue-adhesive terpolymer coating may enable tension-free implant and repair, enhance cellular infiltration/ingrowth and tissue integration by physical abutment, reduce risk of migration, possibly reduce development of seroma, tissue trauma, pain and post surgical adhesions and/or reduce or even remove the need for additional means of mechanical attachment, e.g. suturing, clipping or stapling, particularly in areas of restricted access, and may improve its other properties such as haemostasis through physical retention, rather than overlay and temporary packing.

The device exhibits good initial adhesion to the tissue to which it is applied (and may thus be described as “self-adhesive”), and furthermore remains well-adhered to the tissue over a longer timescale. Without wishing to be bound by any theory, it is believed that the improvement in initial adhesion of the device to the tissue is attributable to electronic bonding of the terpolymer to the tissue, and this is supplemented or replaced by covalent chemical bonding between the tissue-reactive functional groups of the terpolymer and the tissue, in particular between amine and/or thiol groups on the tissue surface and the tissue-reactive functional groups of the terpolymer.

Initial adhesion of the terpolymer coating to the tissue surface is believed to be due to Van der Waals forces and/or hydrogen bonding between the terpolymer and the tissue surface. On contact with the tissue surface the terpolymer becomes hydrated, thereby causing reaction between the tissue-reactive functional groups and the underlying tissue surface. It may also be advantageous, in select cases, to wholly or partially hydrate the device prior to application (e.g. in saline solution) thereby facilitating reaction on application to the tissue surface between the tissue reactive functional groups of the terpolymer and the underlying tissue surface. Such reactions between the tissue-reactive functional groups and the underlying tissue result in high adhesion between the device and the tissue surface. The device may absorb physiological fluids (as a consequence of application onto exuding tissue surfaces), and any additional solutions used to hydrate the sheet following application (such fluids can be commonly used solutions used in surgical irrigation), becoming more compliant and adherent to the tissue surface.

Another advantage of the device according to the invention is that it is supplied to the user and applied to the tissue as a preformed article.

In addition, because the device is, until it contacts the tissue surface or is hydrated immediately prior to application, essentially inactive, the device is not prone to premature reaction and as a result its shelf-life may be considerable, e.g. more than six months when stored appropriately at room temperature.

The terpolymer may be used to modify a wide variety of implantable devices, to produce improved devices according to the present invention. For example, the modification of proprietary graft products by application of the terpolymer has been shown to provide a significant performance benefit. The application of terpolymer significantly improves the adhesion characteristics with no significant change in the handling or flexibility of the product.

### DETAILED DESCRIPTION OF THE INVENTION

### Abbreviations

AAc acrylic acid
AIBN azo-iso-butyronitrile
DPBS Dulbecco's phosphate buffered saline
DCC dicyclohexylcarbodiimide
DCM dichloromethane
DCU dicyclohexylurea
DMF dimethylformamide
ePTFE expanded polytetrafluoroethylene
IPA iso-propanol
M_{w} weight average molecular weight
MeOH methanol
NHS N-hydroxysuccinimide
NVP N-vinyl pyrrolidone
ORC oxidised regenerated cellulose
PGA:TMC poly(glycolide:trimethylene carbonate) copolymer
poly(VP-AAc) copolymer of vinyl pyrrolidone and acrylic acid
poly(VP-AAc-AAc(NHS)) terpolymer of vinyl pyrrolidone, acrylic acid and acrylic acid NHS ester
SIS small intestine submuccosa

### Nature of the Terpolymer Coating

The implantable device of the present invention bears a coating on at least part of the external surface, which coating consists of a terpolymer of vinyl pyrrolidone, acrylic acid and activated acrylic acid, and optionally a colouring agent.

Preferably the coating consists essentially of a terpolymer of vinyl pyrrolidone, acrylic acid and activated acrylic acid. By “essentially” is meant more than 90%, preferably more than 95%, more preferably more than 96%, 97%, 98%, 99%, more preferably more than 99.5%, more preferably more than 99.9%.

Thus, the coating consists of a terpolymer of vinyl pyrrolidone, acrylic acid and activated acrylic acid at a concentration greater than 90%, preferably greater than 95%, more preferably 96%, 97% 98% and 99%, more preferably more than 99.5%, more preferably more than 99.9% by weight of the coating.

In some embodiments, the coating may be said to consist of the terpolymer, optionally with a colouring agent.

By “activated” is meant in the context of the present invention that some of the carboxyl groups are derivatised with tissue-reactive functional groups.

By “tissue-reactive functional groups” is meant functional groups capable of reacting with other functional groups present in the tissue surface so as to form covalent bonds with the tissue. Tissues generally consist partly of proteins, which commonly contain thiol and primary amine moieties. Many functional groups such as imido ester, p-nitrophenyl carbonate, NHS ester, epoxide, isocyanate, acrylate, vinyl sulfone, orthopyridyl-disulfide, maleimide, aldehyde, iodoacetamide, and others, may react with thiols or primary amines, and therefore constitute “tissue-reactive functional groups”. As used herein, the term NHS or NHS ester is intended to encompass not only N-hydroxysuccinimide itself, but also derivatives thereof in which the succinimidyl ring is substituted. An example of such a derivative is N-hydroxysulfosuccinimidyl and salts thereof, particularly the sodium salt, which may increase the solubility of the tissue-reactive material.

Tissue-reactive functional groups that may be of utility in the present invention are any functional groups capable of reaction (under the conditions prevalent when the formulation is applied to tissue, i.e. in an aqueous environment and without the application of significant amounts of heat or other external energy) with functional groups present at the surface of the tissue. The latter class of functional group includes thiol and amine groups, and tissue-reactive functional groups therefore include groups reactive to thiol and/or amine groups. Examples are:
imido ester;
p-nitrophenyl carbonate;
NHS ester;
epoxide;
isocyanate;
acrylate;
vinyl sulfone;
orthopyridyl-disulfide;
maleimide;
aldehyde; and
iodoacetamide.

NHS ester is a particularly preferred tissue-reactive functional group, i.e. the preferred activated acrylic acid is acrylic acid N-hydroxysuccinimide ester.

Only some of the acrylic acid carboxyl groups in the terpolymer will be activated to form tissue-reactive functional groups. Clearly the ratio of activated acrylic acid to acrylic acid in the terpolymer denotes the fraction of acrylic acid carboxyl groups that are activated.

In practice, the currently preferred methods for manufacture of the terpolymer comprise the following steps:

(a) preparing a solution of a copolymer of vinyl pyrrolidone and acrylic acid; and
(b) activating a portion of the acrylic acid carboxyl groups to produce a terpolymer solution.

The acid content of the vinyl pyrrolidone and acrylic acid copolymer solution (a) may be calculated by titration against sodium hydroxide solution, e.g. 1.0M NaOH. It is then possible to accurately determine the amount of reagent required to functionalize a desired percentage of the carboxyl acrylic acid groups.

For example, in preferred embodiments, poly(VP-AAc-AAc(NHS)) terpolymer may be produced by converting the carboxyl groups on poly(VP-AAc) to NHS esters by reaction with NHS in the presence of DCC. If the acid content of the poly(VP-AAc) is determined (in moles), the proportion of acid groups converted to tissue-reactive groups may be controlled by adding the desired mole percent of NHS. As used herein, the notation poly(VPₓ-AAc_{y}-AAc(NHS)_{z}) indicates the molar percentages of the three different monomer residues or derivatives present in the terpolymer For instance, poly(VP₅₀-AAc₂₅-AAc(NHS)₂₅) represents a terpolymer containing 50% vinyl pyrrolidone residues, 25% acrylic acid residues, and 25% NHS-activated acrylic acid residues.

Preferred terpolymers for use in the present invention have approximately one-half of the acrylic acid carboxyl groups activated. Thus, the ratio of acrylic acid and activated acrylic acid is approximately 50:50.

It is preferred that the ratio of vinyl pyrrolidone to acrylic acid and activated acrylic acid together is approximately 50:50.

Thus, in particularly preferred embodiments, the coating of the implantable device is a terpolymer of vinyl pyrrolidone, acrylic acid and activated acrylic acid in the ratio of approximately 50:25:25.

The terpolymer most preferably has a weight average molecular weight M_{w} that is greater than 100,000, more preferably greater than 120,000, 140,000, 160,000, 180,000 or 200,000. The M_{w} of the terpolymer may be greater than 300,000, or greater than 400,000. The M_{w} may be less than 500,000, but may be greater. Terpolymers with relatively high weight average molecular weight M_{w}, i.e. M_{w} greater than 100,000, more preferably greater than 120,000, 140,000, 160,000, 180,000 or 200,000, are found to confer particularly useful properties.

It may be desirable to include an additive in the terpolymer composition in order to colour the terpolymer coating. This may be particularly useful for embodiments of the invention that are only partially coated. For example, where the implantable device is a flat substrate that is coated on one side, it may be useful to distinguish the tissue adhesive coated surface from the uncoated surface. A chromophore dye may be used as a colouring agent, e.g. methylene blue.

Thus, embodiments of the invention in which the terpolymer coating is coloured may be manufactured by a method which method comprises the following steps:

(a) preparing a solution of a copolymer of vinyl pyrrolidone and acrylic acid;
(b) activating a portion of the acrylic acid carboxyl groups to produce a terpolymer solution;
(c) adding methylene blue to the terpolymer solution (b); and
(d) applying the coloured terpolymer solution (c) to a medical device.

Methylene blue may be added to the terpolymer solution at a maximum concentration of 1% w/v, preferably at a maximum concentration of 0.1% w/v, more preferably a maximum concentration of 0.05% w/v, most preferably a maximum concentration of 0.025% w/v.

### Nature of the Device

The present invention is thought to have wide application for many types of medical device.

Application of the terpolymer coating may provide a product with the properties required for implantation and use for a particular application.

More likely, the device is an implantable device with proven use for particular applications, but whose scope of application and performance benefits would be improved by modification of the device with the terpolymer coating.

If the surface of the device is smooth, e.g. a device having a smooth surgical steel exterior, then full encapsulation of the product may be required, in order that the coating remains adequately attached during use.

It is preferred to use devices having a surface that is not perfectly smooth, such that coating may wholly or partially encapsulate parts of the device or may fill interstices in the surface of the device thereby aiding physical attachment of the coating.

So, for instance, the invention may be particularly useful in the coating of mesh-type products, fibrous products, fabrics or the like.

There may be some cross-linking reaction between the terpolymer coating and the medical device to which it is applied, particularly if the device comprises a biomaterial having groups that react with the activated tissue-reactive functional groups of the terpolymer. Such reaction may aid attachment of the terpolymer coating.

One group of implantable devices that are particularly suitable for the present invention are graft products. Suitable graft and physically applied products include but are not limited to the following:
Small intestine submuccosa (Surgisis® Soft Tissue Graft, Cook Biotech Inc)
ePTFE (GORE DUALMESH®, W.L Gore & Associates Inc)
Poly(glycolide:trimethylene carbonate) copolymer (PGA:TMC) (GORE RESOLUT® ADAPT® Regenerative Membrane, W.L Gore & Associates Inc)
Polyester (Mersilene™, Ethicon Inc.) and polypropylene (Prolene, Ethicon Inc) meshes and sheeting
Oxidised regenerated cellulose gauze (Surgicel®, Ethicon Inc)
Pericardium (Peri-strips Dry, Synovis Life Technologies Inc)
Collagen (TissuFleece E, Baxter AG and Lyostypt®, B Braun)
Co-polymers of lactic and glycolic acid, including poly lactic acid (Surgi-Wrap™, MAST Biosurgery) and polyglycolic acid (Dexon Mesh™, United States Surgical), polyglactin (Vicryl, Ethicon Inc)

Furthermore, non-absorbable metal constructs, including but not limited to tantalium, stainless steel and titanium, in the form of sheets, meshes, clothes and complex shapes such as pacemakers may be coated.

### Manufacture of the Implantable Device

Implantable devices according to the invention may be prepared by any convenient method of applying the coating to the device. For example, the terpolymer coating may be applied to all or part of the external surface of a device by casting the formulation over the device, dipping of the devices in liquid formulations or by spraying the device with the liquid formulation.

This is followed by drying to remove solvent, preferably at a relatively low temperature, and preferably under reduced pressure. Drying is preferably carried out at a relatively low temperature to avoid cross-linking of the functional groups that would reduce their availability for reaction with the groups on the surface of tissue to which the device is application, and would therefore reduce the adhesive properties of the implantable device. It may also be important to keep the drying temperature low so that the heat does not alter the coated substrate, in particular when the coated substrate comprises biological material, e.g. growth factors or other proteins. Drying is preferably carried out at temperature between 20° and 50° C., depending on the nature of the substrate and its sensitivity to heat.

The coating may be applied in a single operation. Alternatively, if a thicker coating is required, the coating may be built up by the application of successive layers.

### Adhesion Testing

Sufficiency of the degree of initial adhesion of a sheet to the tissue, by the bioadhesive polymer(s), can be quantitatively determined in vitro, for example by performing an adhesion strength test. This test is performed by allowing the sheet to adhere to a suitable substrate (secured in a fixed position), while the sheet itself is physically attached at a separate point to the load of a tensile testing apparatus, positioned so that, prior to the test, the sheet is not under load. The load cell is moveable along an axis substantially perpendicular to that along which the substrate is positioned. The test involves movement of the load cell away from the substrate, at a constant predetermined rate, until the sheet detaches from the substrate. The output of the test is a quantitative measure of the energy of adhesion for that sheet, i.e. the cumulative amount of energy required to break the interaction between the sheet and the substrate to which it is adhered. A suitable cumulative energy of adhesion for the sheet according to the invention would be not less than 0.5 mJ.

### BRIEF DESCRIPTION OF DRAWING

FIG. 1 illustrates the synthesis of poly(VP-AAc-AAc(NHS)) terpolymer.

### DETAILED DESCRIPTION OF PREFERRED EMBODIMENTS

The invention will now be described in greater detail, by way of illustration only, with reference to the following Examples. Example 1 describes a method for preparing the terpolymer. Example 2 describes the characterisation of the product of Example 1, and the manner in which its properties can be varied by changes in purification conditions. Examples 3-7 describe the manufacture of implantable medical devices according to the invention by application of the terpolymer to commercially available graft products, the handling properties of those devices, and the results of qualitative and quantitative adhesion testing.

### Example 1

### Synthesis of poly(VP₅₀-AAc₂₅-AAc(NHS)₂₅) terpolymer

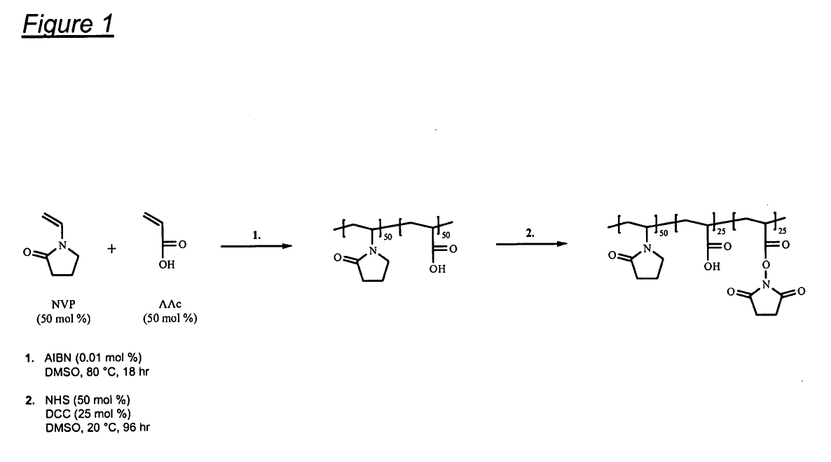

The reaction is shown schematically in FIG. 1.

2000 ml of deoxygenated DMSO is heated to 80° C. 121.3 g (1.09 moles) of NVP and 78.7 g (1.09 moles) of AAc are added to the DMSO followed by 0.04 g (2.44×10⁻⁴ moles) of AIBN. The reaction is left at 80° C. for 17-19 hours and then allowed to cool to room temperature. 125.6 g (1.09 moles) of NHS is dissolved in the polymer solution followed by the addition of 112.6 g (0.545 moles) of DCC dissolved in 225 ml of DMF. The reaction is left stirring at room temperature for 96 hours. The reaction by-product, DCU, is removed by filtration under reduced pressure using a sintered glass filter. The polymer is isolated by mixing with 2000 ml of IPA followed by precipitation from 13000 ml of diethyl ether followed by filtration. The polymer is washed three times in 2500 ml of diethyl ether and then dried at 40° C. under reduced pressure.

The polymer is purified further to remove trace amounts of contaminants by a Soxhlet extraction using IPA.

The Soxhlet extracted polymer is purified further by preparing a 6% w/v solution in DCM/MeOH (15/4 w/v) and then precipitation from a 50-fold excess of diethyl ether, followed by subsequent washing in diethyl ether. The purified polymer is dried at 40° C. under reduced pressure.

### Example 2

### Characterisation of poly(VP-AAc-AAc(NHS)) terpolymer

It is found that the molecular weight of the product of Example 1 is dependent on the duration of the Soxhlet extraction. This in turn affects the viscosity of the product. Table 1 illustrates the dependence of molecular weight and viscosity of the polymer on the duration of the Soxhlet extraction performed in Example 1.

The measurements were performed as follows:

### Molecular Weight Analysis

The samples were analysed in duplicate by GPC in DMF solvent containing 0.1% LiBr. The system was calibrated using PEG/PEO standards. System details were:

### Polymer Laboratories LC1150 HPLC Pump

### Viscotek TDA Model 300, Column Oven and Refractive Index Detector

Polymer Laboratories PLGel Mixed B column, with guard column

### Temperature 70° C.

Flow Rate 1.0 mL/min

### Viscosity Test

This procedure is used to determine the relative viscosity of a solution of the polymer product of Example 1. The procedure is based on flow times through a tube of constant diameter and length and is not an absolute measure of molecular weight. The flow time (dependent upon viscosity) of a solution can be used as an indicative measure of molecular weight—a solution with a longer flow time has a greater molecular weight.

A solution of the test sample is prepared at 15 wt % in DMSO and allowed to equilibrate to 25° C. in a water bath. This is drawn into a 10 mL wide tip pipette (Fisherbrand FB51886) and the flow time from 10 mL→2 mL recorded. Samples are tested in triplicate.

TABLE 1

| Soxhlet Run | | |
|---|---|---|
| Time | Viscosity | Molecular Weight |
| (hour) | (Flow Time, sec) | (M_{w}) |
| 0 | 11.7 | 92,900 |
| 1 | 19.4 | — |
| 2 | 24.5 | 115,000 |
| 3 | 32.5 | — |
| 4 | 48.8 | — |
| 5 | 96.0 | — |
| 6 | 141.2 | 129,700 |
| 7 | 555.9 | 156,700 |

### Example 3

### Application of poly(VP-AAc-AAc(NHS)) terpolymer to Small Intestine Submuccosa (SIS)

SIS is an extracellular matrix comprising collagen, non-collagenous proteins and other biomolecules.

### 3.1 Assessment of Application/Coating Method and Handling Characteristics

### SIS

Poly(VP₅₀-AAc₂₅-AAc(NHS)₂₅) terpolymer prepared according to Example 1 Methylene Blue

### Dichloromethane/methanol (DCM/MeOH) 15:4 v/v

A solution of terpolymer was prepared at 10% w/v in the 15/4 solvent mix, incorporating a maximum of 0.025% w/v methylene blue. The terpolymer solution was applied to SIS using K-bars to give the following approximate coat weights: 10 mg/cm², 15 mg/cm², 20 mg/cm² and 30 mg/cm².

At all four coat weights the terpolymer solution coated down smoothly and evenly onto the SIS graft, but tended to accumulate within the ridges. The product was dried under vacuum for 24 hours at 20° C. to remove solvent residues. The terpolymer solution did not seep through the graft to the reverse side.

The coated product was slightly stiffer than the uncoated product, but could be flexed repeatedly through 90° without loss of terpolymer from the surface. This stiffness was not evident after hydration of the SIS graft. The light blue coloration provided by the methylene blue assisted with determining the coated side.

It should be noted that the product is designed for testing after a period of hydration. After this pre-treatment there is no evidence of stiffness and the product feels and handles in the same manner as uncoated SIS.

### 3.2 Assessment of Adhesion Characteristics

### Qualitative Adhesion Testing

Sections of the coated product measuring 15×15 mm were firstly hydrated in 0.9% saline for 3 minutes and then placed on porcine liver followed by application of moderate pressure for 30 seconds. The samples were then submerged in DPBS for 10 minutes and 18 hours (overnight). The results are presented in Table 2.

The non-coated product offered no adhesion, whereas those with 10 and 30 mg/cm² of terpolymer showed good to strong adhesion initially. After 18 hours submersion both remained affixed to the liver, and the product with the highest coat weight showing the strongest adhesion. The strength of adhesion was sufficient to lift sections of liver weighing 50-100 g off the bench. The coated products could be forcibly removed by aggressive tugging at which point the failure mode was adhesive, i.e. at the interface of the liver and graft suggesting that the terpolymer coating is well integrated within the SIS graft.

The reverse (non-coated) side offered no adhesion.

After soaking for 18 hours the strength of the adhesion had lessened although the coated graft was still in place. The strength of adhesion was not sufficient to lift the liver off the bench. The mode of failure after soaking tended to be cohesive and resulted in a gel of terpolymer remaining on the liver surface.

TABLE 2
Qualitative adhesion testing of coated SIS grafts

| Product | 10 min adhesion | 18 hour adhesion |
|---|---|---|
| SIS | No adhesion | No adhesion |
| SIS + 10 mg/cm² terpolymer | Good adhesion | Moderate adhesion |
| SIS + 30 mg/cm² terpolymer | Strong, well adhered | Moderate adhesion |

### Quantitative Adhesion Testing

The adhesion of the coated grafts to freshly excised liver tissue was assessed quantitatively using a bespoke rig attached to a Zwick Universal Testing Machine in accordance with a validated in-house method. Details of the method are summarized below.

A small section of tissue (4 cm×4 cm×1 cm (depth)) was prepared and mounted into a purpose-made holder at the base of the test machine. The surface of the tissue was sprayed with water. The test specimen (with sample holder attached to enable subsequent removal) was placed onto the tissue surface with a moderate force to ensure full contact. The material was left on the tissue for 5 minutes and then wholly submerged in water for a further 5 minutes. Whilst holding the tissue in place using a suitable clamp the folded tip of the sample holder was inserted in the grips of the UTM. The sample was positioned appropriately to ensure that the sample was aligned with the grips. The grip was then moved at 180° from the test sample thereby removing the sample from the tissue. The UTM software (Zwick TestXpert ver 9.0) can be used to calculate the energy of adhesion (mJ) of the test material.

The test method relies on a natural substrate, in this case porcine liver, and can thus exhibit variations. Hence a control sample of an adhesive patch of known adhesive strength was run alongside on each occasion to validate the results.

The products were prepared for testing by cutting to 15×15 mm followed by fixation to a specially designed stub. The test was undertaken using a peel rate of 5 mm/min and gives the mean energy of adhesion (quoted in mJ). The adhesion of the samples was tested as follows:
Samples hydrated in 0.9% saline for 3 minutes
Application of the patch/stub to porcine liver with moderate pressure for 10 seconds
Leave for 5 minutes
Submerge in DPBS solution for 5 minutes
Undertake n=6 replicates

The test results for the coated SIS grafts are shown in Table 3. The non-coated product had negligible adhesion, as expected from the earlier qualitative study. When coated with terpolymer, the results showed that the higher coat weight product (30 mg/cm²) exhibited good adhesion. The lower coat weight product gave, as expected, a lower figure but this would probably be sufficient to hold the grafts in place.

TABLE 3
Quantitative adhesion testing of coated SIS

| | Energy of |
|---|---|
| Product | adhesion (mJ) |
| SIS | 0.02 |
| SIS + 10 mg/cm² terpolymer | 2.14 |
| SIS + 15 mg/cm² terpolymer | 3.47 |
| SIS + 20 mg/cm² terpolymer | 6.87 |
| SIS + 30 mg/cm² terpolymer | 9.03 |

### 3.3 Summary

The study shows that poly(VP-AAc-AAc(NHS)) terpolymer can be successfully applied to a SIS graft to produce a self adhesive product. The main points to note are:
The terpolymer does not peel or flake off the coated substrate under regular handling conditions. The coated product is stiffer than the uncoated version but this stiffness is eliminated after the initial pre-hydration in saline.
The adhesive properties of the coated products are classed as good.
The reverse (uncoated) side is non-adhesive.
The coated products remain adhered to porcine liver after submerging in DPBS for 18 hours.

### Example 4

### Application of poly(VP-AAc-AAc(NHS)) terpolymer to ePTFE

ePTFE is a sheet of expanded polytetrafluoroethylene (ePTFE) with a smooth and ridged side.

### 4.1 Assessment of Application/Coating Method and Handling Characteristics

ePTFE Graft
Poly(VP₅₀-AAc₂₅-AAc(NHS)₂₅) terpolymer prepared according to Example 1 Methylene blue

### Dichloromethane/methanol (DCM/MeOH) 15:4 v/v

A solution of terpolymer was prepared at 10% w/v in the 15/4 solvent mix, incorporating a maximum of 0.025% w/v methylene blue. The terpolymer solution was applied to the ePTFE graft using K-bars to give the following coat weights: 10 mg/cm² and 30 mg/cm².

At both coat weights the terpolymer solution coated down smoothly and evenly only to the ePTFE graft, but tended to accumulate within the ridges. The product was dried under vacuum for 24 hours at 20° C. to remove solvent residues. The terpolymer solution did not seep through the graft to the reverse side.

### 4.2 Adhesion Testing

### Qualitative Adhesion Testing

Sections of the coated product measuring 15×15 mm were placed on porcine liver followed by application of moderate pressure for 30 seconds. The samples were then submerged in DPBS for 10 minutes and 18 hours (overnight). The results are presented in Table 4.

The non-coated product offered no adhesion, whereas those with 10 and 30 mg/cm² of terpolymer showed good to strong adhesion initially. After 18 hours submersion both remained affixed to the liver, with the product with the highest coat weight showing the strongest adhesion. The coated products could be forcibly removed by aggressive tugging at which point the failure mode was adhesive, i.e. at the interface of the liver and graft suggesting that the terpolymer coating is well integrated with the ePTFE patch.

TABLE 4
Qualitative adhesion testing of coated ePTFE grafts

| Product | 10 min adhesion | 18 hour adhesion |
|---|---|---|
| ePTFE | No adhesion | No adhesion |
| ePTFE + 10 mg/cm² terpolymer | Good adhesion | Moderate-good |
| | | adhesion |
| ePTFE + 30 mg/cm² terpolymer | Strong, well adhered | Good adhesion |

### Quantitative Adhesion Testing

Quantitative adhesion testing was undertaken using the same method as described in section 3.2 of Example 3. The adhesion of the ePTFE grafts was tested as follows:
Application of the patch/stub to porcine liver with moderate pressure for 10 seconds
Submerge in DPBS solution for 5 minutes
Undertake n=6 replicates

The test results for the coated ePTFE grafts are shown in Table 5. The non-coated product had negligible adhesion, as expected from the earlier qualitative study. When coated with terpolymer, the results showed that the higher coat weight product (30 mg/cm²) exhibited good adhesion. The lower coat weight product gave, as expected, a lower figure but this would probably be sufficient to hold the grafts in place.

TABLE 5
Quantitative adhesion testing of coated ePTFE grafts

| | Energy of |
|---|---|
| Product | adhesion (mJ) |
| ePTFE | 0.02 |
| ePTFE + 10 mg/cm² terpolymer | 3.29 |
| ePTFE + 30 mg/cm² terpolymer | 12.47 |

### 4.3 Summary

The ePTFE graft with 30 mg/cm² coating showed the best results for the ePTFE samples tested. The coating appeared to be even and homogenous and showed no signs of delamination or cracking on handling. This sample was well adhered to porcine liver after submersion in DPBS solution over night.

### Example 5

### Application of poly(VP-AAc-AAc(NHS)) terpolymer to poly(qlycolide:trimethylene carbonate) copolymer (PGA:TMC)

This material is a resorbable thick (>1 mm) web made from a copolymer of glycolide and trimethylene carbonate.

### 5.1 Assessment of Application/Coating Method and Handling Characteristics

PGA:TMC graft
Poly(VP₅₀-AAc₂₅-AAc(NHS)₂₅) terpolymer prepared according to Example 1 Methylene blue

### Dichloromethane/methanol (DCM/MeOH) 15:4 v/v

A solution of terpolymer was prepared at 25% w/v in the 15/4 solvent mix, incorporating a maximum of 0.025% w/v methylene blue. The terpolymer solution was applied to the PGA:TMC graft using K-bars to give the following coat weights: 10 mg/cm², 50 mg/cm² and 75 mg/cm².

The PGA:TMC graft has a porous, fibrous structure and this had to be considered during the coating. It was important to ensure that the adhesive coating did not penetrate the entire graft and make the reverse side adhesive. This was controlled by optimising the concentration and hence the viscosity of the terpolymer solution applied. At 25% w/v the terpolymer solution coated down smoothly and evenly onto the PGA:TMC graft. The product was dried under vacuum for 24 hours at 20° C. to remove solvent residues. It could be seen that the terpolymer solution had not seeped through the graft to the reverse side.

There were no differences in the handling characteristics of the non-coated or coated products. Apart from the blue coloration the only appearance difference was a light curling of the coated patch, presumable due to shrinkage upon drying of the terpolymer layer. The coated product could be flexed repeatedly through 90° without loss of polymer from the surface.

### 5.2 Assessment of Adhesion Characteristics

### Qualitative Adhesion Testing

Sections of the coated product measuring 15×15 mm were placed on porcine liver followed by application of moderate pressure for 30 seconds. The samples were then submerged in DPBS for 10 minutes and 18 hours (overnight). The results are presented in Table 6.

TABLE 6
Qualitative adhesion testing of coated PGA:TMC grafts

| Product | 10 min adhesion | 18 hour adhesion |
|---|---|---|
| PGA:TMC | No adhesion | No adhesion |
| PGA:TMC + 30 mg/cm² | Good adhesion | Good-moderate adhesion |
| terpolymer | | |
| PGA:TMC + 50 mg/cm² | Not tested | Not tested |
| terpolymer | | |
| PGA:TMC + 75 mg/cm² | Good adhesion | Good-moderate adhesion |
| terpolymer | | |

The non-coated product offered no adhesion, whereas those with 30 and 75 mg/cm² of terpolymer showed good to strong adhesion initially (the 50 mg/cm² was not tested). After 18 hours submersion both remained affixed to the liver, with both products showing similar adhesive forces. PGA:TMC is a stiffer/less flexible product and although well adhered the impression given to the user is that it is relatively easy to peel off the tissue surface.

The coated products could be forcibly removed by aggressive tugging at which point the failure mode was adhesive, i.e. at the interface of the liver and graft suggesting that the adhesive polymer coating is well integrated with the PGA:TMC patch.

### Quantitative Adhesion Testing

Quantitative adhesion testing was undertaken on the coated PGA:TMC products as described in Example 3. The results are presented in Table 7.

The non-coated product had negligible adhesion, as expected from the earlier qualitative study. Even though the PGA:TMC grafts had varying coating weights of terpolymer applied to them, all exhibited similarly strong adhesion to porcine liver, exceeding the adhesive strength of commonly used surgical sealants whose energy of adhesion (tested under the same conditions is consistently less than 0.5 mJ. This may have been due to the coating penetrating the sample and leaving a similar thickness of terpolymer at the surface of the graft.

The adhesion of the reverse side of the coated PGA:TMC grafts was found to be negligible and so concludes that the terpolymer does not penetrate the entirely of the sample and leaves an anti-adhesive backing.

TABLE 7
Quantitative adhesion testing of coated PGA:TMC grafts

| | Energy of |
|---|---|
| Product | adhesion (mJ) |
| PGA:TMC | 0.04 |
| PGA:TMC + 30 mg/cm² terpolymer | 3.63 |
| PGA:TMC + 50 mg/cm² terpolymer | 4.89 |
| PGA:TMC + 75 mg/cm² terpolymer | 20.78 |

### 5.3 Summary

The PGA:TMC graft coated with 75 mg/cm² showed the best results from the range of PGA:TMC samples tested. The coating appeared to be homogenous and did not penetrate the entirety of the sample, ensuring that the reverse side of the product was non-adhesive. The PGA:TMC graft was found to be a stiff material and did not exhibit great flexibility before coating with terpolymer and so the addition of terpolymer did not appear to affect its mechanical performance. The coating appeared well adhered to the graft and showed no signs of delamination or cracking during handling. This sample was well adhered to porcine liver after submersion in DPBS solution over night and gave good adhesion results, but lower results compared to the ePTFE grafts (Example 4), probably due to the poorer flexibility of the product.

### Example 6

### Application of poly(VP-AAc-AAc(NHS)) terpolymer to a Polyester Hernia Mesh

The mesh selected is a polyethylene terephthalate fibre (polyester) mesh used for herniorrhaphy.

### 6.1 Assessment of Application/Coating Method and Handling Characteristics

Poly(VP₅₀-AAc₂₅-AAc(NHS)₂₅) terpolymer prepared according to Example 1 Polyester hernia mesh
Methylene blue

Due to the porous nature of the mesh, it was decided dip coating offered the best method for applying terpolymer. A stainless steel tray was placed at a shallow angle to allow the solution to pool. The patch was slowly pulled through the solution using forceps and held until dry.

The mesh dip coated in 2.5% w/v solution remained flexible and approximately ¾ of the open mesh structure being maintained with ¼ of the holes being filled with a thin film of terpolymer. The 5.0% w/v coated samples were stiffer than desired but the coating appeared more homogenous. All the holes of the mesh were filled with terpolymer film except for a small hole in the centre of the film. The mesh dip coated in 10% w/v terpolymer solution was very stiff and had no porosity remaining. The sample resembled a film of terpolymer encasing a mesh. The mesh dip coated in 2.5% w/v terpolymer solution gave the best handling performance and appearance.

### 6.2 Assessment of Adhesion Characteristics

Quantitative adhesion testing was undertaken in accordance with the method described in Example 3 as follows:
Application of the patch/stub to porcine liver with moderate pressure for 30 seconds
Leave for 5 minutes
Submerge in DPBS solution for 5 minutes
Undertake n=6 replicates

The results of the quantitative adhesion testing are shown in Table 8.

TABLE 8
Quantitative adhesion testing on terpolymer coated polyester mesh

| | Poly(VP₅₀-AAc₂₅-AAc(NHS)₂₅) terpolymer | Adhesion/ |
|---|---|---|
| Substrate | coat weight | mJ |
| polyester | dip coated in 2.5% w/v terpolymer soln | 1.15 ± 0.73 |
| mesh | | |
| polyester | dip coated in 5.0% w/v terpolymer soln | 1.93 ± 0.35 |
| mesh | | |
| polyester | dip coated in 10.0% w/v terpolymer soln | 3.73 ± 1.32 |
| mesh | | |

### 6.3 Summary

Due to the material being a mesh and the entirety of the product requiring coating, the product was dip coated. The optimum concentration of dip coating solution was found to be 2.5% terpolymer solution. The terpolymer coating appeared to cover and adhere well to the mesh but a small proportion of the mesh holes were filled in by a film of terpolymer. The quantitative adhesion testing of this coated mesh gave relatively low figures for the adhesion, but the qualitative adhesion testing showed that the adhesion was sufficient to hold the mesh in place. The low adhesion figures were probably due to the low surface area of the mesh and the thin coating applied.

### Example 7

### Application of poly(VP-AAc-AAc(NHS)) terpolymer to Oxidised Regenerated Cellulose (ORC)

ORC can be prepared in a number of formats, from a soft fleece to an open mesh structure and is utilised as anabsorbable haemostat.

### 7.1 Assessment of Application/Coating Method and Handling Characteristics

### ORC

Poly(VP₅₀-AAc₂₅-AAc(NHS)₂₅) terpolymer prepared according to Example 1 Methylene Blue

### Dichloromethane/methanol (DCM/MeOH) 15:4 v/v

### Test Coatings

The coating of ORC was initially investigated by using a 300 μm K-bar and at 7.5% and 25% w/v concentration in DCM/MeOH. The coated samples show that the 7.5% penetrated the sample but the 25% w/v solution remained on one side with no penetration.

### Optimisation

Further optimisation was carried out using a 300 μm K-bar and terpolymer solutions at 15%, 17.5% and 20% w/v concentration (containing 0.1% methylene blue). As the concentration was increased, less of the solution penetrated the product. The 20% w/v solution coated with a 300 μm K-Bar was found to remain on one side of the mesh without penetrating the sample. However, the handling performance of the ORC coated with 20% w/v solution with a 300 μm K-bar was found to be stiffer than required, and demonstrated a degree of curling (due to the physical properties of terpolymer. Therefore, a coating applied with a 50 μm K-Bar using a 20% solution was found to be more flexible and provide the desired handling properties. The thinner terpolymer coating had a slight green tint, rather than the usual blue from the methylene blue. This latter coating was deemed to be the optimum and was taken forward for adhesion testing.

### 7.2 Assessment of Adhesion Characteristics

The adhesion of the terpolymer coated ORC was assessed qualitatively by preparing the samples in the same manner as the polyester samples (see Example 6).

### 50 μm K-Bar

The adhesion of ORC coated with 20% w/v terpolymer solution using a 50 μm K-Bar was carried out and compared with untreated product. The results are shown in Table 9.

TABLE 9
Qualitative adhesion testing on terpolymer coated oxidised
regenerated cellulose

| | Poly(VP₅₀-AAc₂₅-AAc(NHS)₂₅) | |
|---|---|---|
| Substrate | terpolymer coat weight | Adhesion/mJ |
| ORC | no coating | 0.16 ± 0.08 |
| ORC | coated with terpolymer (20% w/v soln + | 1.67 ± 074 |
| | 50 μm K-Bar) | |

The adhesion of the coated ORC was found to be quite low but this not surprising as the terpolymer layer may have been quite thin taking into account the thickness of coating applied and the potential for the coating to soak into the sample. However, the results from the pre-clinical study (see below) showed that samples prepared with the same coating thickness performed well in vivo, so for this product, the adhesion was found to be sufficient.

### Adhesion Testing on a Wider Range of Coat Weights

A solution of terpolymer at 20 wt % was used, as this has been shown not to fully penetrate the material and cause the reverse side to become adhesive. Using a range of K-bars from 24 to 300 μm the mass of terpolymer deposited on the material does increase, but not by the expected amounts. This could be due to the undulating and/or porous nature of the substrate. Both the energy of adhesion of ORC to porcine liver and tensile strength of the coated device increase with greater coat weight, albeit moderately, whereas the flexibility of the coated device decreased quite noticeably over the test range. It has also been shown that no terpolymer is lost from the coated device upon vigorous flexing, folding and rolling.

Qualitative adhesion testing was also undertaken after 10 minutes application and after soaking overnight in DPBS. The adhesion was scored as moderate after t=0 minutes and moderate/good after t=16 hours. As is clearly shown in Table 10, the strength of the coated device outweighs the adhesive properties of the terpolymer, however the adhesion which is present is more than capable of holding the device in place.

TABLE 10
Qualitative adhesion testing on terpolymer coated ORC for a range
of coat weights

| | | Mass of | Tensile | Energy of |
|---|---|---|---|---|
| | K | Terpolymer | Strength | Adhesion |
| Substrate | Bar | mg/cm² | MPa | mJ |
| ORC | — | — | 14.12 (±2.10) | 0.34 (±0.20) |
| ORC | 24 | 3.8 | 13.83 (±0.79) | 2.13 (±0.42) |
| ORC | 50 | 4.4 | 14.95 (±1.09) | 2.36 (±1.82) |
| ORC | 80 | 4.2 | 15.40 (±0.67) | 1.52 (±0.22) |
| ORC | 100 | 4.8 | 16.01 (±0.95) | 2.05 (±0.43) |
| ORC | 200 | 6.8 | 18.49 (±1.43) | 2.37 (±0.43) |
| ORC | 300 | 7.5 | 18.30 (±1.80) | 3.53 (±1.50) |

### 7.3 Pre-Clinical Assessment

The haemostatic and adhesive properties of oxidised regenerated cellulose (ORC) coated with terpolymer (20% w/v solution using a 50 μm K-Bar). This was compared with uncoated product in a porcine liver punch biopsy model.

The wounds assessed were a) 6 mm punch biopsy created in the central lobe, resulting in a low/moderate bleed and b) rectangular injuries measuring approximately 1.5×2×0.25 mm prepared on the same lobe.
When applied as a patch, ORC coated with terpolymer has consistently achieved haemostasis in punch biopsy and surface excision injuries—originally generating low-moderate rate blood losses;
In comparison, treatment of the surface excision injury with a patch of standard ORC was not capable of achieving haemostasis;
The short term adhesion (<hour) of standard ORC is poor when applied flat onto bleeding sites. ORC originally applied over a punch biopsy was found to be free floating after 45 minutes within the abdominal cavity;
Coating ORC with terpolymer enables the device to be successfully applied as a patch. This combination device adheres to the liver for a period of at least 45 minutes (within the abdominal cavity), indicative that the coated material will remain in place adhered to the target site supporting the tissue prior to resorption over a period of time.

### 7.4 Summary

The coating which gave the optimum handling performance was applied using a 20% w/v of terpolymer solution and a 50 μm K-Bar. This coating was also found to remain on one side of the mesh without penetrating through to the other side. The coated oxidised regenerated cellulose showed good adhesion in vivo and also improved haemostatic properties (by physical means) compared with the untreated product.

1. An implantable medical device, at least part of the external surface of which bears a coating consisting essentially of a terpolymer of vinyl pyrrolidone, acrylic acid and activated acrylic acid, and optionally a colouring agent.

2. A device as claimed in claim 1, wherein the activated acrylic acid is derivatised with a functional group selected from the group consisting of imido ester, p-nitrophenyl carbonate, epoxide, isocyanate, acrylate, vinylsulfone, orthopyridyl-disulfide, maleimide, aldehyde, iodoacetamide, and N-hydroxysuccinimide ester groups.

3. A device as claimed in claim 1 or claim 2, wherein the activated acrylic acid is acrylic acid N-hydroxysuccinimide ester.

4. A device as claimed in any preceding claim, wherein approximately one-half of the acrylic acid carboxyl groups in the terpolymer are activated.

5. A device as claimed in any preceding claims, wherein the ratio of vinyl pyrrolidone to acrylic acid and activated acrylic acid together is 50:50.

6. A device as claimed in any preceding claim, wherein the colouring agent is a chromophore dye.

7. A device as claimed in claim 7, wherein the chromophore dye is methylene blue.

8. A device as claimed in any preceding claim, which device is an implantable graft product.

9. A method of preparing a device according to claim 1, which method comprises the following steps:
(a) preparing a solution of a copolymer of vinyl pyrrolidone and acrylic acid;
(b) activating a portion of the acrylic acid carboxyl groups to produce a terpolymer solution; and
(c) applying the terpolymer solution to a medical device.

10. A method as claims in claim 9, wherein the ratio of vinyl pyrrolidone to acrylic acid in the copolymer is 50:50.

11. A method as claimed in claim 9 or claim 10, wherein the acrylic acid carboxyl groups are activated in step (b) by derivatisation with a functional group selected from the group consisting of imido ester, p-nitrophenyl carbonate, epoxide, isocyanate, acrylate, vinylsulfone, orthopyridyl-disulfide, maleimide, aldehyde, iodoacetamide, and N-hydroxysuccinimide ester groups.

12. A method as claimed in claim 11, wherein the acrylic acid carboxyl groups are activated in step (b) by derivatisation with N-hydroxysuccinimide ester groups.

13. A method as claimed in any one of claims 9 to 12, wherein approximately one-half of the acrylic acid carboxyl groups are activated in step (b).

14. A method as claimed in any one of claims 9 to 13, which comprises the additional step of adding an colouring agent to the terpolymer solution before application of the terpolymer solution to a medical device.
